(19) United States
(12) Patent Application Publication
(10) Pub. No.: US 2025/0268574 A1
(43) Pub. Date: Aug. 28, 2025

(54) ENDOCAVITY ULTRASONIC PROBE

(71) Applicant: Shenzhen Mindray Bio-Medical Electronics Co., Ltd., Shenzhen (CN)

(72) Inventors: Weitao HUANG, Shenzhen (CN); Ming Tang, Shenzhen (CN)

(21) Appl. No.: 19/049,756

(22) Filed: Feb. 10, 2025

(30) Foreign Application Priority Data
Feb. 24, 2024 (CN) 202410167133.1

(51) Int. Cl. A61B 8/00; A61B 8/12

(52) U.S. Cl. CPC A61B 8/4461; A61B 8/12; A61B 8/483

(57) ABSTRACT

A hand pose recognition method is performed by a computer device, including: acquiring a current frame of a multi-lens video of a target object; performing hand detection on a first view of the current frame to obtain a first lens detection result; performing hand estimation on a second view of the current frame to obtain a second lens estimation result; removing, from the hand detection boxes in the first view and the hand estimation boxes in the second view, redundant boxes corresponding to redundant hands, and then performing hand joint point recognition on remaining boxes to obtain two-dimensional joint points; converting the two-dimensional joint points into three-dimensional joint points in a three-dimensional hand coordinate system; and converting the three-dimensional joint points in the three-dimensional hand coordinate system into three-dimensional joint points of the current frame in a world coordinate system according to pose estimation parameters corresponding to the current frame.

### CROSS-REFERENCE TO RELATED APPLICATIONS

This application claims priority to Chinese Patent Application No. 202410167133.1, entitled “ENDOCAVITY ULTRASONIC PROBE” filed with the China National Intellectual Property Administration on Feb. 24, 2024, which is incorporated herein by reference in their entirety.

### FIELD OF THE TECHNOLOGY

The present application relates to the field of medical devices, and more particularly, to endocavity ultrasonic probes.

### BACKGROUND OF THE DISCLOSURE

Endocavity ultrasonic probes are employed within the body cavities of humans or animals to obtain images of those cavities. In pursuit of enhanced visualization, these probes are commonly designed as 3D or 4D ultrasonic probes capable of generate three-dimensional (3D) images. The transducers of these 3D or 4D probes oscillate around an axis, enabling them to acquire two-dimensional (2D) ultrasonic images (B-mode images) from various oscillation angles. Subsequently, these 2D images are processed and combined to reconstruct a 3D image.

The field of view (FOV) of the resultant 3D image is determined by the oscillation angle of the transducer and the imaging angle of the 2D ultrasonic image it captures. A larger oscillation angle or a wider imaging angle contributes to a broader FOV in the ultimate 3D image. Nevertheless, present-day 3D and 4D ultrasonic probes generally have transducers with a maximum oscillation angle of approximately 120°, and the imaging angle of the 2D ultrasonic image is less than 180°. Consequently, the FOV of the 3D images generated by these probes is insufficient for applications requiring extensive visualization, such as in cases of obesity.

### SUMMARY

The present disclosure provides an endocavity ultrasonic probe capable of increasing the 2D ultrasonic scanning range of the transducer, thereby expanding the FOV of the resultant 3D image.

The present disclosure also provides an endocavity ultrasonic probe capable of broadening the scanning angle of the transducer, thereby enhancing the FOV of the resultant 3D image.

In accordance with a first aspect of the present disclosure, an endocavity ultrasonic probe provided in some embodiments may include:
a transducer, wherein the transducer comprises a backing layer, a transducer element layer, a matching layer and a lens layer, the transducer element layer comprises a plurality of transducer elements, the backing layer is disposed on the positive side of the transducer elements, the matching layer is disposed on the negative side of the transducer elements, the lens layer is disposed on the side of the matching layer that is opposite to the transducer elements, the transducer element layer is arranged in the form of an arc surface having a first axis as its central axis, and an angle between the two ends of the transducer element layer and the first axis is greater than 180°;
a transducer support, wherein the transducer support comprises a swing axle, and the transducer support is configured to support the transducer;
a base, wherein the transducer support is rotatably mounted on the base; and
a transducer drive component, wherein the transducer drive component comprises a transmission member and a drive member, the transmission member is configured to drive the swing axle and the transducer to swing relative to the base, and the drive member is configured to provide a force for driving the swing;
wherein a surface of a side of the backing layer away from the transducer element layer forms an accommodating space, the swing axle is located in the accommodating space and integrally formed with the backing layer or fixed on the backing layer, the two ends of the swing axle the corresponding transducer element layer are separated by at least a portion of the backing layer, a vertical distance between a line connecting the two ends of the transducer element layer and the apex of the arc surface is greater than a vertical distance between the axis of the swing axle and the apex of the arc surface, and the swing axle is rotatably mounted on the base.

According to the endocavity ultrasonic probe shown in the embodiments, the angle a between the two ends of the transducer element layer and the first axis A is greater than or equal to 180°, which expands the scanning range of the transducer element layer and, consequently increases the scanning angle of resulting 2D ultrasonic images. Moreover, due to the swing axle being disposed in the accommodating space of the backing layer, the overall volume occupied by the backing layer and the swing axle is reduced, as the space is fully utilized.

In some embodiments, the accommodating space has two concave and oppositely arranged mounting spaces, and the two ends of the swing axle are inserted into and fixed within the corresponding mounting spaces, respectively.

In some embodiments, both ends of the swing axle are fixed within the corresponding mounting spaces by at least one of welding, adhesive bonding, snap fitting, screwing, or fastening.

In some embodiments, both ends of the swing axle are provided with support bases that are either integrally formed with the swing axle or fixedly connected to it, and the two ends of the swing axle are fixed within the corresponding mounting spaces by the support bases.

In some embodiments, the mounting spaces are extended downwards to reach the bottom surface of the backing layer, and the support bases have abutting surfaces that abut against the bottom surface.

In some embodiments, the support bases have L-shaped or T-shaped structures, the two ends of the swing axle are connected to the vertical legs of said structures, and the abutting surfaces are on the horizontal legs of said structures.

In some embodiments, the support bases are fixedly connected to the bottom surface by at least one of welding, adhesive bonding, snap fitting, screwing, or fastening.

In some embodiments, the transducer support further comprises a driven wheel disposed coaxially on the swing axle; the base is provided with a swing bracket extending towards the swing axle and a housing surrounding the swing bracket, wherein the swing bracket is protruded relative to the housing to form its protruding part that has a swing support member, the swing axle is rotatably mounted on the swing support member, and the dimension of the protruding part is at least two-thirds of the radius of the driven wheel; and the transmission member is mounted on the driven wheel to drive the driven wheel, such that the swing axle and the transducer swing on the swing support member.

In some embodiments, the driven wheel is disposed eccentrically with respect to the lengthwise center of the swing axle along its longitudinal axis.

In some embodiments, at least two swing brackets are provided; and the swing axle is provided with a limiting formation that prevents it from moving longitudinally relative to the swing bracket.

In some embodiments, the first axis extends through the swing axle.

In some embodiments, the first axis is perpendicular to the axis of the swing axle.

In some embodiments, the lens layer has an arc-shaped structure that matches the arc surface, with a radius of curvature of R; and a difference between the vertical distance between a line connecting the two ends of the transducer element layer and the apex of the arc surface and the vertical distance between the axis of the swing axle and the apex of the arc surface is at least 0.5R.

In accordance with a second aspect of the present disclosure, an endocavity ultrasonic probe provided in some embodiments may include:
a transducer for transmitting and receiving ultrasonic signals;
a transducer support, wherein the transducer is mounted on the transducer support, and the transducer support comprises a swing axle and a driven wheel that is coaxially disposed on the swing axle;
a base, wherein the transducer support is rotatably connected to the base, the base is provided with a swing bracket extending towards the swing axle and a housing surrounding the swing bracket, the swing bracket is protruded relative to the housing to form its protruding part that has a swing support member, the swing axle is rotatably mounted on the swing support member, and a vertical distance between the axis of the swing axle and the uppermost edge of the housing is configured to allow the transducer to swing on the base with a swing angle of at least 180°; and
a transducer drive component, wherein the transducer drive component comprises a transmission member and a drive member, the transmission member is configured to drive the swing axle and the transducer to swing relative to the base, and the drive member is configured to provide a force for driving the swing.

According to the endocavity ultrasonic probe shown in the embodiments, a vertical distance is designed between the axis of the swing axle and the uppermost edge of the housing, allowing the transducer to swing on the base with an angle of at least 180°. This increases a distance between the structures on the swing axle (such as the driven wheel) and the uppermost edge of the housing, enabling the scanning angle of the transducer, and consequently expanding the FOV of the 3D images obtained by the transducer.

In some embodiments, the vertical distance between the axis of the swing axle and the uppermost edge of the housing is at least two-thirds of the radius of the driven wheel.

In some embodiments, the driven wheel is disposed eccentrically with respect to the lengthwise center of the swing axle along its longitudinal axis.

In some embodiments, at least two swing brackets are provided; and the swing axle is provided with a limiting formation that prevents it from moving longitudinally relative to the swing brackets.

In some embodiments, the transducer comprises a backing layer, a transducer element layer, a matching layer and a lens layer; the transducer element layer comprises a plurality of transducer elements, the backing layer is disposed on the positive side of the transducer elements, the matching layer is disposed on the negative side of the transducer elements, the lens layer is disposed on the side of the matching layer that is opposite to the transducer elements; the transducer elements are arranged in a form of an array to form the transducer element layer, and the transducer element layer is arranged in a form of an arc surface having a first axis as its central axis.

In some embodiments, the first axis is perpendicular to the axis of the swing axle.

In accordance with a third aspect of the present disclosure, an endocavity ultrasonic probe provided in some embodiments may comprise:
a transducer, wherein the transducer comprises a backing layer, a transducer element layer, a matching layer and a lens layer, the transducer element layer comprises a plurality of transducer elements, the backing layer is disposed on a positive side of the transducer elements, the matching layer is disposed on a negative side of the transducer elements, the lens layer is disposed on a side of the matching layer that is opposite to the transducer elements, the transducer element layer is arranged in the form of an arc surface having a first axis as its central axis, and an angle between the two ends of the transducer element layer and the first axis is at least 180;
a transducer support for supporting the transducer;
a base, wherein the transducer support is rotatably connected to the base; and
a transducer drive component, wherein the transducer drive component comprises a transmission member and a drive member, the transmission member is configured to drive the transducer support and the transducer to swing relative to the base, and the drive member is configured to provide a force for driving the swing.

According to the endocavity ultrasonic probe shown in the embodiments, due to the angle between the two ends of the transducer element layer and the first axis being greater than or equal to 180°, the scanning range of the transducer element layer can be enlarged, thus improving the scanning angle of resulting 2D ultrasonic images.

In some embodiments, the surface of the backing layer away from the transducer element layer forms an accommodating space.

### BRIEF DESCRIPTION OF THE DRAWINGS

To describe the technical solutions in the embodiments of this application more clearly, the following briefly describes the accompanying drawings required for describing the embodiments or conventional technologies. It is clear that the accompanying drawings in the following descriptions show merely some embodiments of this application, and a person of ordinary skill in the art may still derive other drawings from these disclosed accompanying drawings without creative efforts.

FIG. 1 is a schematic diagram of the appearance of an endocavity ultrasonic probe according to some embodiments of the present disclosure, wherein the cable part of the endocavity ultrasonic probe is omitted therefrom;

FIG. 2 is a sectional view of an endocavity ultrasonic probe according to some embodiments of the present disclosure;

FIG. 3 is a sectional view of a transducer according to some embodiments of the present disclosure;

FIG. 4 is a 3D schematic diagram of a transducer according to some embodiments of the present disclosure;

FIG. 5 is a sectional view of a transducer element layer and a backing layer according to some embodiments of the present disclosure;

FIG. 6 is a sectional view, taken along the axis of the swing axle, of the base, the transducer support and the transducer that have been assembled together according to some embodiments of the present disclosure;

FIG. 7 is a sectional view, taken along a direction perpendicular to the axis of the swing axle, of the base, the transducer support and the transducer that have been assembled together according to some embodiments of the present disclosure;

FIG. 8 is a schematic diagram of assembling the transducer support with the backing layer according to some embodiments of the present disclosure;

FIG. 9 is a schematic diagram showing the base and the transducer support according to some embodiments of the present disclosure; and

FIG. 10 is a sectional view of a transducer element layer together with a backing layer according to another embodiment of the present application.

### DESCRIPTION OF EMBODIMENTS

The present disclosure will be further described in detail below through specific embodiments with reference to the accompanying drawings. Common or similar elements are referenced with like or identical reference numerals in different embodiments. Many details described in the following embodiments are for better understanding the present disclosure. However, those skilled in the art can realize with minimal effort that some of these features can be omitted in different cases or be replaced by other elements, materials and methods. For clarity some operations related to the present disclosure are not shown or illustrated herein so as to prevent the core from being overwhelmed by excessive descriptions. For those skilled in the art, such operations are not necessary to be explained in detail, and they can fully understand the related operations according to the description in the specification and the general technical knowledge in the art.

In addition, the features, operations or characteristics described in the specification may be combined in any suitable manner to form various embodiments. At the same time, the steps or actions in the described method can also be sequentially changed or adjusted in a manner that can be apparent to those skilled in the art. Therefore, the various sequences in the specification and the drawings are only for the purpose of describing a particular embodiment, and are not intended to be an order of necessity, unless otherwise stated one of the sequences must be followed.

The serial numbers of components herein, such as “first”, “second”, etc., are only used to distinguish the described objects and do not have any order or technical meaning. The terms “connected”, “coupled” and the like here include direct and indirect connections (coupling) unless otherwise specified.

An endocavity ultrasonic probe disclosed herein, capable of functioning as either a 3D ultrasonic probe or a 4D ultrasonic probe, obtains 3D images of cavities within an object under examination. The FOV of these images is correlated with both the scanning angle of the transducer and the 2D ultrasonic scanning range of the transducer. However, in present-day 3D and 4D ultrasonic probes, the 2D ultrasonic scanning range of the transducer cannot be expanded beyond 180°, and the maximum scanning angle of the transducer is limited to approximately 120°. As a result, the FOV of the 3D images cannot be further expanded.

After carefully analyzing the structure of existing endocavity ultrasonic probes, the inventor has identified the reasons why the field of view (FOV) of 3D images cannot be further expanded. Firstly, this limitation is imposed by the application environment. The external dimensions of the endocavity ultrasonic probes, such as their circumferential size and outer diameter, cannot be further enlarged because excessive large external dimensions can readily cause discomfort when used within human or animal bodies, consequently restricting the design of the internal space of each probe. Secondly, the design of the swing mechanism for the transducers in the endocavity ultrasonic probes lack optimization, limiting their ability to achieve wider angles for both 2D ultrasonic imaging and the swing motion.

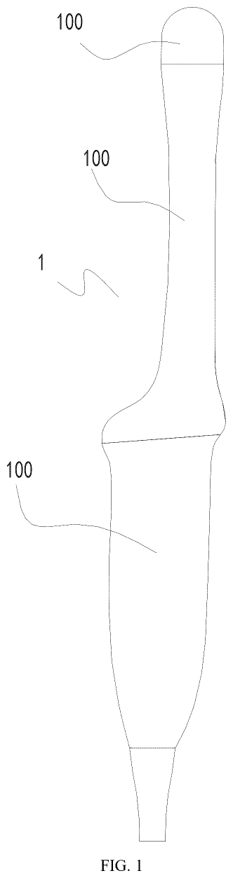

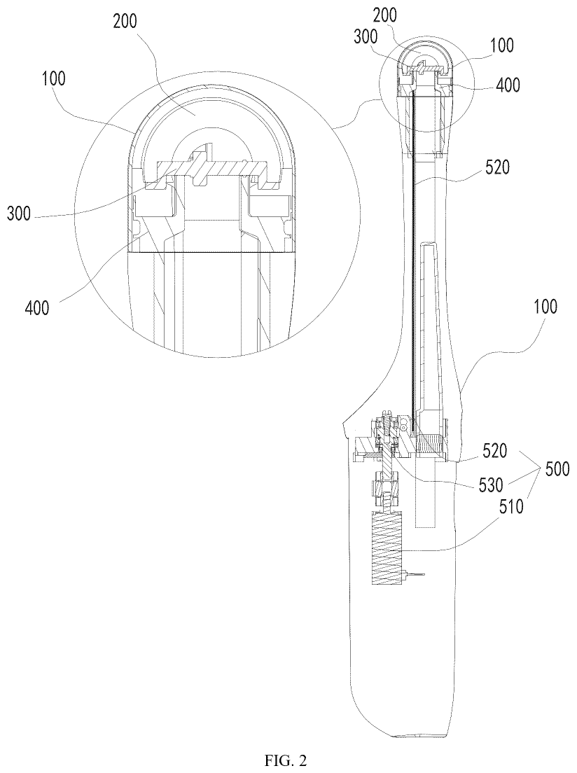

In consideration of current circumstances, further refinements to the swing mechanism of the transducer have been introduced in the present disclosure. Referring to FIG. 1 and FIG. 2, in some embodiments, the endocavity ultrasonic probe 1 may include an outer shell 100, a transducer 200, a transducer support 300, a base 400, a transducer drive component 500 and a control unit (not shown in the figures). The outer shell 100 forms an enclosed cavity for installation, within which the transducer 200, the transducer support 300, the base 400, the transducer drive component 500 and the control unit are all installed.

The transducer 200 may be configured to transmit and receive ultrasonic signals under the control of the control unit. The control unit may be configured to process the received ultrasonic signals to obtain ultrasonic images. The transducer 200 is mounted on the transducer support 300, which is rotatably connected to the base 400. The transducer drive component 500 may include a transmission member 520 configured to drive the transducer support 300 and the transducer component 200 to swing relative to the base 400, and a drive member 510 configured to provide a force for the swing. Under the control of the control unit, the transducer 200 can acquire 2D ultrasonic images at various swing angles, and the control unit can then combine these 2D images from different angles to form a 3D image.

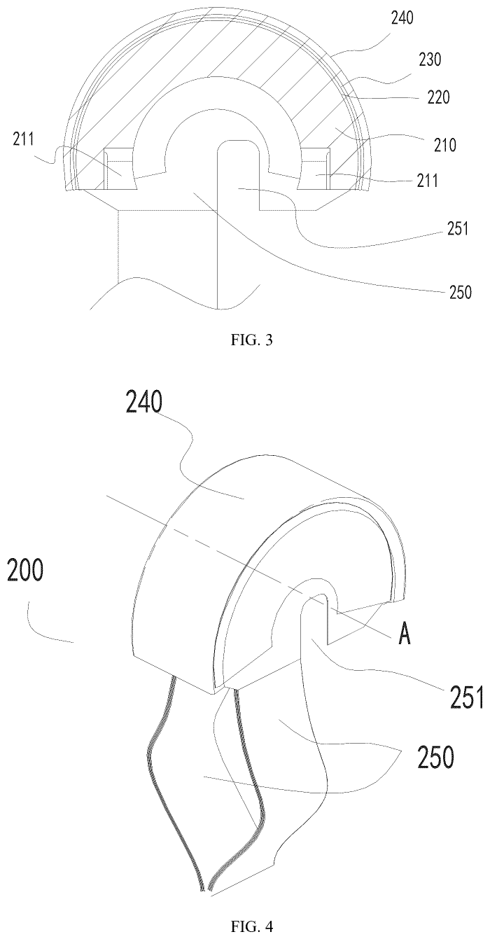

Referring to FIG. 3 and FIG. 4, in some embodiments, the transducer 200 may include a backing layer 210, a transducer element layer 220, a matching layer 230 and a lens layer 240. The transducer element layer 220 may include a plurality of transducer elements which can be arranged in an array to form a planar array, or in a line to form a linear array, or a convex array, or in other arrangements. The lens layer 240, the matching layer 230, the transducer element layer 220 and the backing layer 210 may be arranged in sequence, with the backing layer 210 disposed on the positive side of the transducer elements, the matching layer 230 disposed on the negative side of the transducer elements, and the lens layer 240 disposed on the side of the matching layer 230 away from the transducer elements.

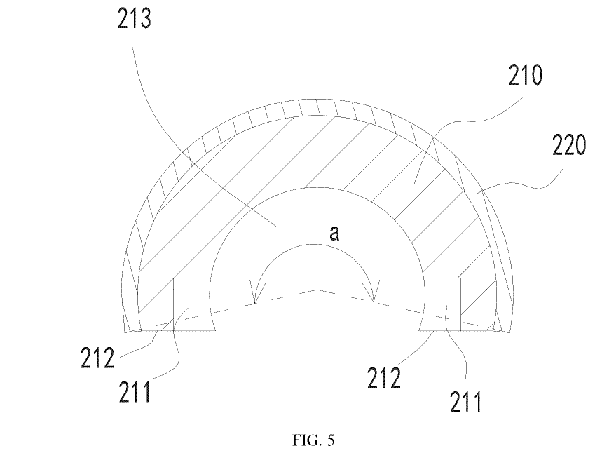

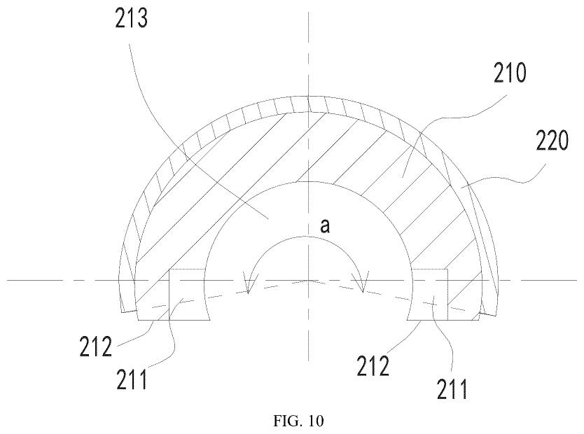

The scanning angle of 2D ultrasonic images obtained by the transducer 200 is inherently linked to the distribution range of the transducer elements. To broaden this scanning angle, referring to FIG. 4 and FIG. 5, in some embodiments, the transducer element layer 220 may be arranged in the form of an arc surface having the first axis A as its central axis, with the angle a between the two ends of the transducer element layer 220 and the first axis A being at least 180° (i.e. greater than or equal to) 180°. This design enlarges the scanning range of the transducer element layer 220, and thus improving the scanning angle of the resulting 2D ultrasonic images. Of course, to support the transducer element layer 220, referring to FIG. 5 and FIG. 10, in some embodiments, the surface of the backing layer 210 facing the transducer element layer 220 is also arranged in the form of an arc surface having the first axis A as its central axis, and the angle between the two ends of the backing layer 210 and the first axis A is at least 180°, allowing for a larger region on the backing layer 210 to accommodate the transducer elements. This enables the angle a between the distribution region of the transducer elements and the first axis A to be at least 180°.

During the research into the swing mechanism of the transducer 200 in the existing endocavity ultrasonic probe 1, it was discovered that it is challenging to configure the angle a between the two ends of the transducer element layer 220 and the first axis A to be greater than or equal to 180°. This difficulty arises because the transducer support 300 obstructs the expansion of the transducer element layer 220, thereby limiting the further enlargement of its area.

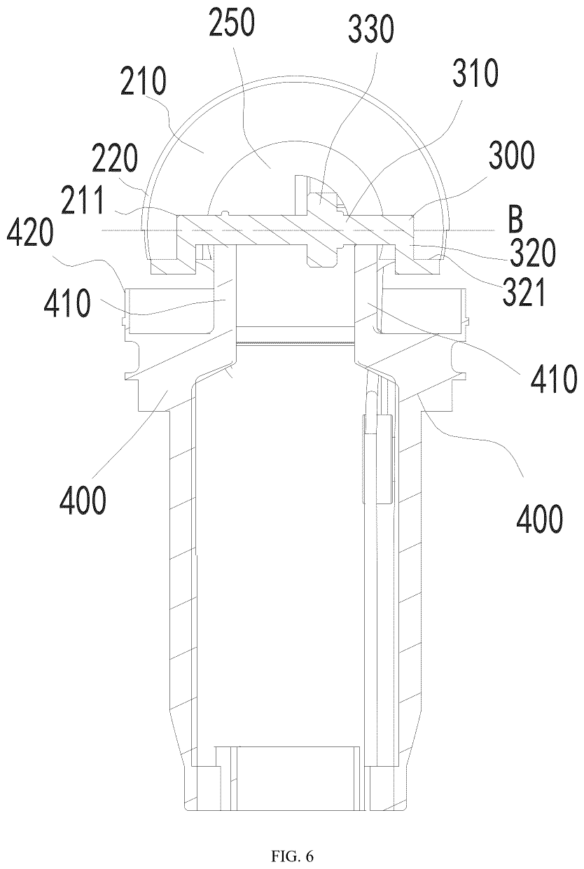

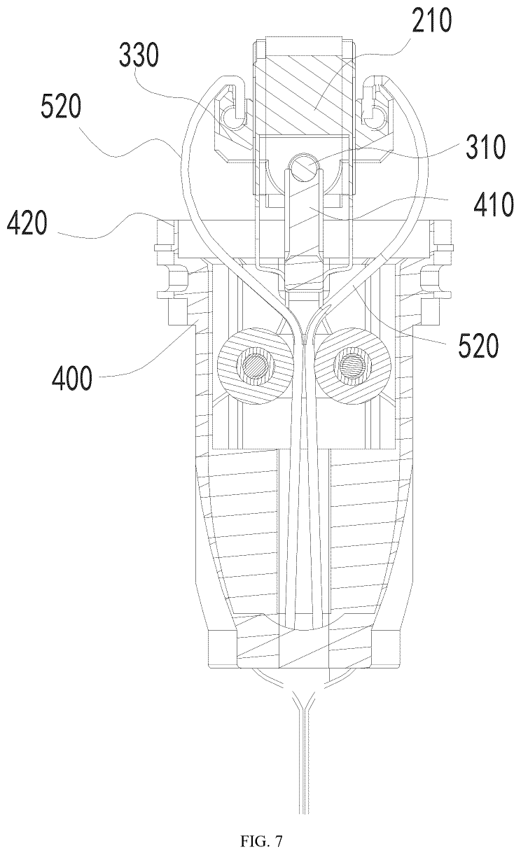

In this regard, referring to FIGS. 6 and 7, in some embodiments, the transducer support 300 is configured to support the transducer 200. The transducer support 300 may be rotatably mounted on the base 400 to facilitate a swingable design. Specifically, the transducer support 300 may include a swing axle 310 that may be rotatably mounted on the 400. Driven by the drive member 510, the transmission member 520 drives the swing axle 310 and the transducer 200 to swing relative to the base 400. The surface of the backing layer 210 away from the transducer element layer 220 (i.e. the side facing the swing axle 310) may form an accommodating space 213. The swing axle 310 is disposed within this space and is cither integrally formed with the backing layer 210 or fixed to it. The fixation may involve directly fixing the swing axle 310 to the backing layer 210, or it may also involve indirectly fixing the swing axle 310 to the backing layer 210 through other components. By fully utilizing the accommodating space 213 of the backing layer 210, the swing axle 310 is effectively embedded within the backing layer 210, thus reducing the overall volume occupied by the backing layer 210 and the swing axle 310.

At least a portion of the backing layer 210 may separate the two ends of the swing axle 310 from their corresponding transducer element layer 220, ensuring that the ends of the swing axle 310 do not interfere with the arrangement of the transducer elements. This allows the side of the backing layer 210 away from the swing axle 310 to serve as a complete surface for placing more transducer elements and thereby expanding the range and angle of the 2D ultrasonic images obtained by the transducer 200. The vertical distance between a line connecting the two ends of the transducer element layer 220 and the apex of the arc surface may be greater than or equal to the vertical distance between the axis B of the swing axle 310 and the apex of the arc surface. The line connecting the two ends of the transducer element layer 220 refers to the line connecting the lowest points of the two ends of the array layer 220 in the orientation as shown in FIG. 6. In the orientation shown in FIG. 6, the lowest points of the two ends of the transducer element layer 220 may be level with or below the axis B of the swing axle 310, ensuring that the angle between the two ends of the transducer element layer 220 and the first axis A is at least 180°. Furthermore, when the vertical distance between a line connecting the two ends of the transducer element layer 220 and the apex of the arc surface is greater than the vertical distance between the axis B of the swing axle 310 and the apex of the arc surface, meaning that the lowest points of the two ends of the transducer element layer 220 are below the axis B of the swing axle 310, it is ensured that the angle between the two ends of the transducer element layer 220 and the first axis A is greater than 180°.

In some embodiments, the lens 240 may be configured as an arc-shaped structure that matches the arc surface of the transducer element layer 220; and when the radius of curvature of this arc-shaped structure is R, the difference between the vertical distance between a line connecting the two ends of the transducer element layer 220 and the apex of the arc surface and the vertical distance between the axis B of the swing axle 310 and the apex of the arc surface is greater than or equal to 0.5R. This ensures that the angle a can be set to cover at least 180°.

Furthermore, in some embodiments, the first axis A extends through the swing axle 310, ensuring that during the swing motion of the transducer 200 along with the swing axle 310, the distance between the outermost point of the transducer 200 and the axis B of the swing axle 310 is approximately equal to the distance between the outermost point of the transducer 200 and the first axis A. As a result, the region traced by the outermost point of the transducer 200 during its swinging approximates a hemispherical shape, which helps to minimize the space required for the swinging motion of the transducer 200 and avoids increasing the volume of the endocavity ultrasonic probe 1.

In some embodiments, the first axis A is perpendicular to the axis B of the swing axle 310; and in this respect, the distance between the outermost point of the transducer 200 and the axis B of the swing axle 310 is approximately equal to the distance between the outermost point of the transducer 200 and the first axis A. As a result, the region traced by the outermost point of the transducer 200 during its swinging is approximately hemispherical, which further reduces the space required for the swinging motion of the transducer 200 and avoids increasing the volume of the endocavity ultrasonic probe 1.

Furthermore, to enhance the fixation between the backing layer 210 and the swing axle 310, as illustrated in FIGS. 5 and 6, in some embodiments, the accommodating space 213 has two concave, oppositely disposed mounting spaces 211. The two ends of the swing axle 310 are inserted and fixed into the corresponding mounting spaces 211. This design not only conceals the swing axle 310 within the backing layer 210 but also maximizes the region on the side of the backing layer 210 away from the swing axle 310 for arranging the transducer elements, thereby expanding their distribution range.

The fixed connection between the swing axle 310 and the backing layer 210 can be achieved using any method that is applicable to ultrasonic probes in existing technology. For example, in some embodiments, both ends of the swing axle 310 are fixed within the corresponding mounting spaces 211 by at least one of welding, adhesive bonding, snap fitting, screwing, or fastening.

Furthermore, referring to FIGS. 5, 6, 8 and 9, in some embodiments, the transducer support 300 further comprises support bases 320, and both ends of the swing axle 310 is secured within corresponding mounting spaces 211 by means of the support bases 320.

In some embodiments, both ends of the swing axle 310 may be integrally molded with or securely attached to the support bases 320. The attachment between the swing axle 310 and the support bases 320 can be implemented using any method commonly applied to ultrasonic probes in existing technology. For example, in some embodiments, the attachment can be achieved by at least one of welding, adhesive bonding, snap fitting, screwing, or fastening.

Further, with reference to FIGS. 5, 6, 8, and 9, in some embodiments, the mounting spaces 211 extend downwards to reach the bottom surface 212 of the backing layer 210 (wherein the bottom surface 212 is defined based on the orientation shown in FIG. 6). The support bases 320 have abutting surfaces 321 that abut against the bottom surface 212. The abutting surfaces are designed to contact and support the bottom surface of the backing layer, thereby supporting the transducer. Specifically, the abutting surfaces 321 is securely attached to the backing layer 210 using any method commonly applied to ultrasonic probes in existing technology. For example, in some embodiments, the attachment can be achieved by at least one of welding, adhesive bonding, snap fitting, screwing, or fastening.

The support bases 320 transfers the fixed position of the swing axle 310 and the backing layer 210 from within the mounting spaces 211 to the bottom surface 212 of the backing layer 210, thereby reducing processing difficulty and facilitating easier manufacturing. Furthermore, compared to the confined mounting spaces 211, the abutting surface 321 and the bottom surface 212 of the backing layer 210 offer a larger area for making a secure connection, resulting in a more stable fixation. The abutting surface 321 not only serves to securely connect with the backing layer 210 but also functions to support the backing layer 210. The abutting surface 321 and the bottom surface 212 of the backing layer 210 form a pair of mutually adapted surfaces, which can be, but are not limited to, a pair of mutually adapted flat surfaces, curved surfaces, or folded surfaces.

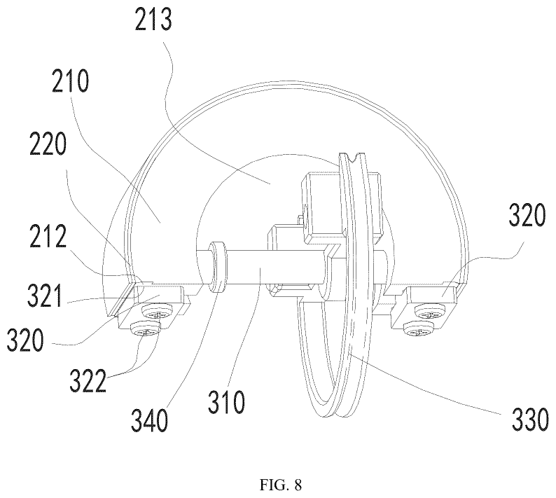

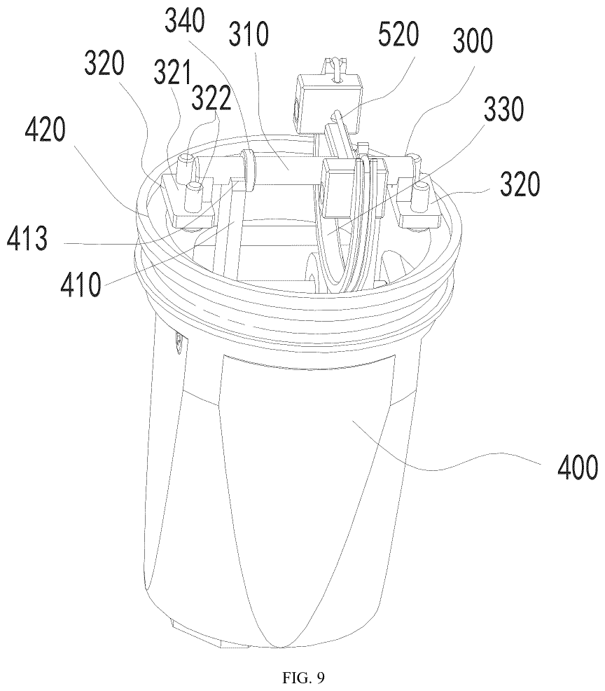

Referring to FIGS. 8 and 9, in some embodiments, the abutting surfaces 321 is secured by at least one positioning screw 322 which engages with a positioning hole on the bottom surface 212 of the backing layer 210, aiding in positioning and fixation of the support bases 320 and the backing layer 210 for more precise installation of the support bases 320 and the swing axle 310. To further prevent rotation, two or more positioning screws can be provided on each abutting surface 321.

Further, referring to FIGS. 5, 6, 8, and 9, in some embodiments, each support bases 320 has an L-shaped structure, with both ends of the swing axle 310 connected to the vertical leg of the L-shaped structure, and the abutting surfaces 321 disposed on the horizontal leg of the L-shaped structure. The surface of the horizontal leg facing the backing layer 210 serves as the abutting surfaces 321. Similarly, in other embodiments, the support bases 320 may be of a T-shaped structure, with both ends of the swing axle 310 connected to the vertical leg of the T-shaped structure, and the abutting surfaces 321 disposed on the horizontal leg of the T-shaped structure. It shall be understood that the terms “horizontal” and “vertical” used here refer to the respective segments of the letter-shaped structures, without implying any strict orientation in physical space beyond their relative positions within the structure itself.

Further, referring to FIG. 9, in some embodiments, two support bases 320 are provided, which are respectively disposed at both ends of the swing axle 310 and fixed to the backing layer 210 from the opposite sides of the backing layer, thus enhancing the stability of the fixed connection among the support bases 320, the swing axle 310 and the backing layer 210.

Further, referring to FIG. 9, in some embodiments, the swing axle 310 and the support bases 320 are integrally formed as a single piece for case of manufacture. Alternatively, in other embodiments, the swing axle 310 and the support bases 320 may be separately manufactured and then fixedly connected either directly or indirectly.

Furthermore, referring to FIGS. 6-9, in some embodiments, the transducer support 300 also includes a driven wheel 330 disposed on the swing axle 310, with the driven wheel 330 and the swing axle 310 being coaxially arranged. The base 400 is provided with a swing bracket 410 extending towards the swing axle 310 and a housing 420 surrounding the swing bracket 410. The swing bracket 410 protrudes relative to the housing 420, and the protruding portion of the swing bracket 410 is equipped with a swing support member 413 (see FIG. 9). The swing axle 310 is rotatably mounted on the swing support member 413. The transmission member 520 is mounted on the driven wheel 330 to drive the driven wheel 330, the swing axle 310 and the transducer 200 to swing on the swing support member 413.

In some more specific embodiments, referring to FIGS. 2 and 7, the transmission member 520 is a drive rope that is looped around the driven wheel 330. The drive member 510 can pull the two ends of the transmission member 520 respectively through a transmission mechanism 530. As shown in FIG. 2, the drive member 510 is a motor that, through forward and reverse rotation, can pull the two ends of the transmission member to move, thereby causing the driven wheel 330 to swing to the left or right (as shown in FIG. 7), and subsequently driving the swing axle 310 and the transducer 200 to swing.

Further, referring to FIG. 4, in some embodiments, the transducer 200 may also include a lead-out circuit structure 250 (such as a flexible circuit board) for leading out the positive and negative electrodes of the transducer elements. This lead-out circuit structure 250 protrudes from the side of the backing layer 210 and is electrically connected to a control circuit, allowing the control circuit to input electrical signals to the transducer elements and receive electrical signals from them through the lead-out circuit structure 250, thereby controlling the transducer elements.

Due to potential spatial conflicts between the position of the lead-out circuit structure 250 and the position of the driven wheel 330, as shown in FIG. 6, in some embodiments, to better provide space for the lead-out circuit structure 250, the driven wheel 330 is offset from the lengthwise center of the swing axle 310. In this embodiment illustrated in FIG. 6, the driven wheel 330 is positioned closer to the right side of the swing axle 310 as viewed in the figure, such that in the orientation shown in FIG. 6, the swing axle 310 is segmented into a longer section on the left and a shorter section on the right by the presence of the driven wheel 330. Referring back to FIG. 4 in conjunction with FIG. 6, it can be seen that the lead-out circuit structure 250 may extend from the left side of the driven wheel 330 as shown in FIG. 6, thus avoiding the need to divide the lead-out circuit structure 250 into two parts and lead it out from both sides of the driven wheel 330 due to space constraints. Alternatively, in some embodiments, the lead-out circuit structure 250 may have a notch 251 (as shown in FIG. 4) that accommodates the driven wheel 330, allowing it to fit within the space provided by the notch 251.

Further, in some embodiments, there are at least two swing bracket 410, and the swing axle 310 is equipped with a limiting formation that prevents it from moving along its length relative to the swing bracket 410. Referring to FIGS. 8 and 9, in the shown embodiment, the limiting formation is a ridge 340 circumferentially disposed around the swing axle 310. This ridge 340 can restrict axial movement of the swing axle 310 within the swing bracket 410. Of course, in other embodiments, the limiting formation can adopt other configurations or be implemented in other ways, such as using a protrusion.

Further, please refer to FIG. 7, in some embodiments, in a cross-section perpendicular to the axis of the swing axle 310, the driven wheel 330 protrudes from the transducer 200 in the left-right direction. The swing axle 310 is supported on the swing bracket 410 of the base 400, with the housing 420 formed by the base 400 located around the swing axle 310. During the research on the swinging structure of the transducer 200, the inventor also found that, even if the swing bracket 410 protrudes from the housing 420, the driven wheel 330 will gradually approach the upper edge of the housing 420 as the transducer 200 oscillates with the swing axle 310, until it comes into contact with the upper edge of the housing 420. This is one of the reasons why the swing angle of the transducer 200 in the existing swinging structure cannot be further increased. Therefore, in some embodiments of this application, there is a vertical distance between the axis B of the swing axle 310 and the uppermost edge of the housing 420, which allows the swing angle of the transducer 200 on the base 400 to be greater than or equal to 180°, thereby increasing the FOV of the 3D images acquired by the transducer 200. Of course, in this embodiment, the transducer 200 can also be substituted with other existing transducers 200, not solely restricted to those with structures specifically designed to increase the angle of 2D ultrasonic imaging as illustrated in the preceding embodiments.

Further, in some embodiments, to enable the transducer 200 to swing at an angle of 180° or more relative to the base 400, the dimension of the protruding part of the swing bracket 410 protruding from the uppermost edge of the housing 420 is at least two-thirds of the radius of the driven wheel 330. This design ensures that the driven wheel 330 does not collide with the uppermost edge of the housing 420 during swinging, thereby allowing the swing angle of the transducer 200 to reach or exceed 180°.

In some other embodiments, the vertical distance between the axis B of the swing axle 310 and the uppermost edge of the housing 420 is at least two-thirds of the radius of the driven wheel 330. This design can prevent the driven wheel 330 from colliding with the uppermost edge of the housing 420 during the swinging process, thereby enabling the transducer 200 to swing at an angle of 180° or more.

The aforementioned embodiments demonstrate structures for increasing the angle of 2D ultrasonic images obtained by the transducer 200, as well as structures for increasing the swing angle of the transducer 200, thereby expanding the FOV of the 3D images acquired by the transducer 200. Of course, in other embodiments, these structures for increasing the angle of the 2D images and the swing angle of the transducer 200 can also be used independently, and both can effectively broaden the FOV of the 3D images obtained by the transducer 200.

For example, referring to FIGS. 6-9, in some embodiments, an endocavity ultrasonic probe 1 comprises a transducer 200, a transducer support 300, a base 400, and a transducer drive component 500. The transducer 200 is configured to emit and receive ultrasonic signals and mounted on the transducer support 300. The transducer support 300 is provided with a swing axle 310 and a driven wheel 330 coaxially arranged on the swing axle 310. The transducer support 300 is rotatably connected to the base 400. The base 400 includes a swing bracket 410 extending towards the swing axle 310 and a housing 420 surrounding the swing bracket 410. The swing bracket 410 protrudes relative to the housing 420, and the protruding part of the swing bracket 410 is provided with a swing support member 413. The swing axle 310 is rotatably mounted on the swing support member 413. The vertical distance between the axis B of the swing axle 310 and the uppermost edge of the housing 420 is sufficient to allow the transducer 200 to swing at an angle of 180° or more on the base 400. The transducer drive component 500 includes a transmission member 520 and a drive member 510. The transmission member 520 is configured to drive the swing axle 310 and the transducer 200 to swing relative to the base 400, while the drive member 510 provides a force for the swinging motion.

In some embodiments, the vertical distance between the axis B of the swing axle 310 and the uppermost edge of the housing 420 is at least two-thirds of the radius of the driven wheel 330.

Referring to FIGS. 6-9, in some embodiments, along the longitudinal direction of the swing axle 310, the driven wheel 330 is offset from the lengthwise center of the swing axle 310.

Referring to FIGS. 6-9, in some embodiments, there are at least two swing brackets 410, and the swing axle 310 is provided with a limiting formation that prevents the swing axle 310 from moving relative to the swing bracket 410 along its axis.

Referring to FIG. 3, in some embodiments, the transducer 200 includes the backing layer 210, the transducer element layer 220, the matching layer 230 and the lens layer 240. The transducer element layer 220 has a plurality of transducer elements. The backing layer 210 is disposed on the positive side of the transducer elements. The matching layer 230 is disposed on the negative side of the transducer elements. The lens layer 240 is disposed on a side of the matching layer 230 away from the transducer elements. The transducer elements are arranged in an array to form the transducer element layer 220, and the transducer element layer 220 is arranged in the form of an arc surface having the first axis A as its central axis.

In some embodiments, the first axis A is perpendicular to the axis B of the swing axle 310.

The specific examples employed above to illustrate the present invention are for the purpose of facilitating understanding and are not intended to limit the invention. Those skilled in the art to which the present invention pertains, without departing from the basic principles of the present invention, may make various simple deductions, modifications or substitutions.

1. An endocavity ultrasonic probe, comprising:
a transducer, wherein the transducer comprises a backing layer, a transducer element layer, a matching layer and a lens layer, the transducer element layer comprises a plurality of transducer elements, the backing layer is disposed on a positive side of the transducer elements, the matching layer is disposed on a negative side of the transducer elements, the lens layer is disposed on a side of the matching layer that is opposite to the transducer elements, the transducer element layer is arranged in a form of an arc surface having a first axis as its central axis, and an angle between two ends of the transducer element layer and the first axis is greater than 180°;
a transducer support, wherein the transducer support comprises a swing axle, and the transducer support is configured to support the transducer;
a base, wherein the transducer support is rotatably mounted on the base; and
a transducer drive component, wherein the transducer drive component comprises a transmission member and a drive member, the transmission member is configured to drive the swing axle and the transducer to swing relative to the base, and the drive member is configured to provide a force for driving the swing;
wherein a surface of the backing layer away from the transducer element layer forms an accommodating space, the swing axle is located in the accommodating space and integrally formed with the backing layer or fixed on the backing layer, two ends of the swing axle and the corresponding transducer element layer are separated by at least a portion of the backing layer, a vertical distance between a line connecting the two ends of the transducer element layer and an apex of the arc surface is greater than a vertical distance between an axis of the swing axle and the apex of the arc surface, and
the swing axle is rotatably mounted on the base.

2. The endocavity ultrasonic probe according to claim 1, wherein the accommodating space has two concave and oppositely arranged mounting spaces, and the two ends of the swing axle are inserted into and fixed within the corresponding mounting spaces, respectively.

3. The endocavity ultrasonic probe according to claim 2, wherein the two ends of the swing axle are fixed within the corresponding mounting spaces by at least one of welding, adhesive bonding, snap fitting, screwing, or fastening.

4. The endocavity ultrasonic probe according to claim 2, wherein the two ends of the swing axle are provided with support bases that are integrally formed with the swing axle or fixedly connected to the swing axle, and the two ends of the swing axle are fixed within the corresponding mounting spaces by the support bases.

5. The endocavity ultrasonic probe according to claim 4, wherein the mounting spaces are extended downwards to reach a bottom surface of the backing layer, and the support bases have abutting surfaces that abut against the bottom surface.

6. The endocavity ultrasonic probe according to claim 5, wherein the support bases have L-shaped or T-shaped structures, the two ends of the swing axle are connected to vertical legs of said structures, and the abutting surfaces are on horizontal legs of said structures.

7. The endocavity ultrasonic probe according to claim 5, wherein the support bases are fixedly connected to the bottom surface by at least one of welding, adhesive bonding, snap fitting, screwing, or fastening.

8. The endocavity ultrasonic probe according to claim 1, wherein the transducer support further comprises a driven wheel disposed coaxially on the swing axle; the base is provided with a swing bracket extending towards the swing axle and a housing surrounding the swing bracket, wherein the swing bracket is protruded relative to the housing to form its protruding part that has a swing support member, the swing axle is rotatably mounted on the swing support member, and a dimension of the protruding part is at least two-thirds of a radius of the driven wheel; and the transmission member is mounted on the driven wheel to drive the driven wheel, such that the swing axle and the transducer swing on the swing support member.

9. The endocavity ultrasonic probe according to claim 8, wherein the driven wheel is disposed eccentrically with respect to a lengthwise center of the swing axle along its longitudinal axis.

10. The endocavity ultrasonic probe according to claim 9, wherein at least two swing brackets are provided; and the swing axle is provided with a limiting formation that prevents it from moving longitudinally relative to the swing brackets.

11. The endocavity ultrasonic probe according to claim 1, wherein the first axis extends through the swing axle.

12. The endocavity ultrasonic probe according to claim 1, wherein the first axis is perpendicular to the axis of the swing axle.

13. The endocavity ultrasonic probe according to claim 1, wherein the lens layer has an arc-shaped structure that matches the arc surface, with a radius of curvature of R; and a difference between the vertical distance between a line connecting the two ends of the transducer element layer and the apex of the arc surface and the vertical distance between the axis of the swing axle and the apex of the arc surface is at least 0.5R.

14. An endocavity ultrasonic probe, comprising:
a transducer for transmitting and receiving ultrasonic signals;
a transducer support, wherein the transducer is mounted on the transducer support, and the transducer support comprises a swing axle and a driven wheel that is coaxially disposed on the swing axle;
a base, wherein the transducer support is rotatably connected to the base, the base is provided with a swing bracket extending towards the swing axle and a housing surrounding the swing bracket, the swing bracket is protruded relative to the housing to form its protruding part that has a swing support member, the swing axle is rotatably mounted on the swing support member, and a vertical distance between an axis of the swing axle and an uppermost edge of the housing is configured to allow the transducer to swing on the base with an angle of at least 180°; and
a transducer drive component, wherein the transducer drive component comprises a transmission member and a drive member, the transmission member is configured to drive the swing axle and the transducer to swing relative to the base, and the drive member is configured to provide a force for driving the swing.

15. The endocavity ultrasonic probe according to claim 14, wherein the vertical distance between the axis of the swing axle and the uppermost edge of the housing is at least two-thirds of a radius of the driven wheel.

16. The endocavity ultrasonic probe according to claim 14, wherein the driven wheel is disposed eccentrically with respect to a lengthwise center of the swing axle along its longitudinal axis.

17. The endocavity ultrasonic probe according to claim 14, wherein at least two swing brackets are provided; and the swing axle is provided with a limiting formation that prevents it from moving longitudinally relative to the swing brackets.

18. The endocavity ultrasonic probe according to claim 14, wherein the transducer comprises a backing layer, a transducer element layer, a matching layer and a lens layer, the transducer element layer comprises a plurality of transducer elements, the backing layer is disposed on a positive side of the transducer elements, the matching layer is disposed on a negative side of the transducer elements, and the lens layer is disposed on a side of the matching layer that is opposite to the transducer elements; the transducer elements are arranged in a form of an array to form the transducer element layer, and the transducer element layer is arranged in a form of an arc surface having a first axis as its central axis.

19. The endocavity ultrasonic probe according to claim 18, wherein the first axis is perpendicular to the axis of the swing axle.

20. An endocavity ultrasonic probe, comprising:
a transducer, wherein the transducer comprises a backing layer, a transducer element layer, a matching layer and a lens layer, the transducer element layer comprises a plurality of transducer elements, the backing layer is disposed on a positive side of the transducer elements, the matching layer is disposed on a negative side of the transducer elements, the lens layer disposed on a side of the matching layer that is opposite to the transducer elements, the transducer element layer is arranged in a form of an arc surface having a first axis as its central axis, and an angle between two ends of the transducer element layer and the first axis is at least 180°;
a transducer support, wherein the transducer support is configured to support the transducer;
a base, wherein the transducer support is rotatably mounted on the base; and
a transducer drive component, wherein the transducer drive component comprises a transmission member and a drive member, the transmission member is configured to drive the transducer support and the transducer to swing relative to the base, and the drive member is configured to provide a force for driving the swing.
